(19) United States
(12) Patent Application Publication
(10) Pub. No.: US 2014/0276660 A1
(43) Pub. Date: Sep. 18, 2014

(54) FLUID DRAIN TUBE WITH TISSUE OXYGENATION CONTENT SENSOR

(71) Applicant: BeckerSmith Medical, Inc., Los Angeles, CA (US)

(72) Inventor: Jan M. Eckermann, Los Angeles, CA (US)

(73) Assignee: BeckerSmith Medical, Inc., Los Angeles, CA (US)

(21) Appl. No.: 14/209,630

(22) Filed: Mar. 13, 2014

Related U.S. Application Data
(60) Provisional application No. 61/779,833, filed on Mar. 13, 2013.

(51) Int. Cl. A61M 27/00; A61B 5/03

(52) U.S. Cl. CPC A61M 27/00; A61B 5/031
USPC 604/543

(57) ABSTRACT

A catheter for draining body fluid that includes a tube for draining the fluid that has an outside surface, an inside surface, a tip and defines a lumen therein. The tip includes at least one opening therein that is in fluid communication with the lumen, at least a first sensor configured to measure the oxygen partial pressure in brain tissue positioned on the outside surface of the tube at a first location, and at least a first wire having a proximal end and a distal end. The distal end is in electrical communication with the first sensor and the proximal is configured to be in electrical communication with a monitor. The tube and the first sensor can be inserted into a single hole in a patient's cranium.

### CROSS REFERENCE TO RELATED APPLICATIONS

This application claims the benefit of U.S. Provisional Application No. 61/779,833 filed Mar. 13, 2013, which is incorporated herein by reference in its entirety.

### FIELD OF THE INVENTION

The present invention relates to a fluid drain tube, and more particularly to a cerebrospinal fluid (“CSF”) drain catheter or tube with a sensor that measures the partial pressure of oxygen in brain tissue.

### BACKGROUND OF THE INVENTION

In patient's with head trauma, a catheter may be inserted to drain excess fluid from the skull to relieve intracranial pressure. Catheters for CSF drainage are known. For example, see U.S. Pat. No. 6,210,346 to Hall, issued Apr. 3, 2001 and U.S. Pat. No. 5,117,836 to Millar, issued on Jun. 2, 1992, the entireties of which are incorporated by reference herein Furthermore, research shows that prior to the intracranial pressure going up brain tissue oxygenation decreases. Accordingly, devices for monitoring tissue pO2, i.e., the oxygen partial pressure in the tissue, in the brain, have been developed. For example, see U.S. Pat. No. 5,891,100 to Fleckenstein, issued Apr. 6, 1999, U.S. Pat. No. 6,068,743 to Fleckenstein, issued May 30, 2000 and U.S. Pat. No. 6,454,774 to Fleckenstein, issued Sep. 24, 2002, the entireties of which are incorporated by reference herein. Detecting this drop in pressure allows doctors to perform preventative measures prior to a resulting increase in intracranial pressure. Currently, the device for monitoring brain tissue oxygenation levels and the device for drainage of fluid are separate. Therefore, at least two separate holes have to be drilled in the patient's skull. So the patients often have, on the right side of the skull, a hole for ventricular draining, and, on the left side of the skull, a hole for an insert for monitoring brain tissue oxygenation levels. Accordingly, a need exists for a device that provides measurement of the brain/neuro tissue oxygenation content and drainage through a single hole in the skull.

### SUMMARY OF THE PREFERRED EMBODIMENTS

In accordance with a first aspect of the present invention there is provided a catheter for draining body fluid that includes a tube for draining the fluid that has an outside surface, an inside surface, a tip and defines a lumen therein. The tip includes at least one opening therein that is in fluid communication with the lumen, at least a first sensor configured to measure the oxygen partial pressure in brain tissue positioned on the outside surface of the tube at a first location, and at least a first wire having a proximal end and a distal end. The distal end is in electrical communication with the first sensor and the proximal is configured to be in electrical communication with a monitor. The tube and the first sensor can be inserted into a single hole in a patient's cranium. In a preferred embodiment, the catheter further includes a second sensor that is configured to measure the oxygen partial pressure in brain tissue positioned on the outside surface of the tube at a second location. The second wire has a proximal end and a distal end. The distal end is in electrical communication with the second sensor and the proximal end is configured to be in electrical communication with a monitor. The outer surface of the tube has a circumference and preferably, the second location is between approximately 10° and approximately 180° from the first location about the circumference of the outer surface of the tube.

In a preferred embodiment, the catheter further includes a third sensor that is configured to measure the oxygen partial pressure in brain tissue positioned on the outside surface of the tube at a third location. The third wire has a proximal end and a distal end. The distal end is in electrical communication with the third sensor and the proximal end is configured to be in electrical communication with a monitor. Preferably, the third location is between approximately 10° and approximately 180° from the first location about the circumference of the outer surface of the tube.

In a preferred embodiment, the catheter further includes a fourth sensor that is configured to measure the oxygen partial pressure in brain tissue positioned on the outside surface of the tube at a fourth location. The fourth wire has a proximal end and a distal end. The distal end is in electrical communication with the fourth sensor and the proximal end is configured to be in electrical communication with a monitor. Preferably, the second location is approximately 90° from the first location about the circumference of the outer surface of the tube, the third location is approximately 90° from the second location about the circumference of the outer surface of the tube, and the fourth location is approximately 90° from the third location about the circumference of the outer surface of the tube.

In a preferred embodiment, the first location is a first distance from the tip and the second location is a second distance from the tip. The first distance is preferably different than the second distance. In another preferred embodiment, the first location is a first distance from the tip, the second location is a second distance from the tip, and the third location is a third distance from the tip. Preferably, the first, second and third distances are preferably all different. In another preferred embodiment, the first location is a first distance from the tip, the second location is a second distance from the tip, the third location is a third distance from the tip, and the fourth location is a fourth distance from the tip. The first, second, third and fourth distances are preferably all different.

In a preferred embodiment, the first wire is secured to the outside surface of the tube. In another preferred embodiment, the first wire is embedded in the tube, and the first sensor extends outside of the tube. Preferably, the first wire includes an end portion that is movable between a disengaged position where it is positioned adjacent the outer surface of the tube and an engaged position wherein it extends outwardly away from the outer surface of the tube. In a preferred embodiment, each of the first, second, third and fourth wires include an end portion that is movable between a disengaged position where it is positioned adjacent the outer surface of the tube and an engaged position wherein it extends outwardly away from the outer surface of the tube.

In accordance with another aspect of the present invention, there is provided a method of draining fluid through a single opening in a patient's cranium. The method includes the steps of providing a catheter that includes a tube and at least a first sensor for measuring oxygen partial pressure in brain tissue, inserting the tube into the opening in the patient's cranium such that the first sensor is surrounded by the patient's brain tissue, draining fluid from the opening, and measuring the oxygen partial pressure of surrounding brain tissue. In a preferred embodiment, the first sensor includes a first wire in electrical communication therewith that extends through the opening in the patient's cranium. Preferably, the catheter includes a second sensor and a second wire in electrical communication therewith that extends through the opening in the patient's cranium.

Generally, at least one small wire probe or sensor is inserted into the brain that measures tissue oxygenation for the partial pressure of oxygen in the brain together with a drainage tube.

The invention, together with additional features and advantages thereof, may be best understood by reference to the following description.

### BRIEF DESCRIPTION OF THE DRAWINGS

FIG. 1A is a perspective view of a catheter in accordance with a preferred embodiment of the present invention;

FIG. 1B is a cross-sectional view taken along line 1B-1B of FIG. 1A;

FIG. 1C is a cross-sectional view taken along line 1C-1C of FIG. 1A;

FIG. 1D is a cross-sectional view taken along line 1D-1D of FIG. 1A;

FIG. 1E is a cross-sectional view taken along line 1E-1E of FIG. 1A;

FIG. 2A is a perspective view of a catheter in accordance with a preferred embodiment of the present invention;

FIG. 2B is a cross-sectional view taken along line 2B-2B of FIG. 2A;

FIG. 2C is a cross-sectional view taken along line 2C-2C of FIG. 2A;

FIG. 2D is a cross-sectional view taken along line 2D-2D of FIG. 2A;

FIG. 2E is a cross-sectional view taken along line 2E-2E of FIG. 2A;

FIG. 3 is a perspective view of a catheter in accordance with another preferred embodiment of the present invention; and

FIG. 4 is a cross-sectional view of the catheter of FIG. 3 in vivo.

Like numerals refer to like parts throughout the several views of the drawings.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an other embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. Appearances of the phrase “in one embodiment” in various places in the specification do not necessarily refer to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” and “below” used herein are merely for ease of description and refer to the orientation of the components as shown in the figures. It should be understood that any orientation of the components described herein is within the scope of the present invention.

As shown in FIGS. 1A-4, the present invention is a catheter 100 that includes at least one probe or sensor 10 for measuring oxygen partial pressure in the brain tissue that is mounted on or associated with a CSF drain tube 12. The sensor 10 may be mounted on the outside, embedded in or otherwise connected to the drain tube 12. The sensor 10 is positioned on the tube 12 such that it is inside the skull during use.

As shown in FIGS. 1A-2E, in a preferred embodiment, the tube 12 defines a lumen 14 for draining CSF or other body fluids therethrough. The end of the tube 12 includes a tip 16 and a plurality of holes 18 for entry of the CSF fluid into the lumen 14. In a preferred embodiment, the tube 12 includes an array of sensors 10 for measuring oxygen partial pressure and associated wires 22 mounted on the outside thereof. As shown in FIGS. 1A-1B and 2A-2B, in a preferred embodiment, the sensors 10 and wires 22 are positioned around the tube 12 at predetermined intervals (e.g., at twelve, three, six and nine o'clock or 0°, 90°, 180° and 270°). The wires 22 can be secured or attached to the outside 12a of the tube 12 (FIGS. 1A-1E) or they can be embedded in the material of the tube 12 and extend through an opening to the outside thereof (FIGS. 2A-2E).

It will be appreciated that the wires 22 and sensors 10 can be positioned at equal intervals or locations from one another circumferentially or at non-equal intervals or locations. Furthermore, any number of sensors 10 and associated wires 22 is within the scope of the present invention. In an embodiment with two (i.e., first and second) sensors 10 and wires 22, the location of the second sensor 10 and wire 22 is preferably between approximately 10° and approximately 180° about the circumference of the outer surface of the tube 12 from the location of the first sensor 10 and wire 22 (in either direction). In an embodiment with three (i.e., first, second and third) sensors 10 and wires 22, the location of the second sensor 10 and wire 22 is preferably between approximately 10° and approximately 180° about the circumference of the outer surface of the tube 12 from the location of the first sensor 10 and wire 22 (in either direction). The location of the third sensor 10 and wire 22 is also preferably between approximately 10° and approximately 180° about the circumference of the outer surface of the tube 12 from the location of the first sensor 10 and wire 22 (in either direction). In a specific embodiment, moving clockwise about the outer circumference, if the first sensor 20 is located at 0°, the second sensor is preferably located at about 120° and the third sensor is located at about 240°. These examples are provided as though one was viewing the tube 12 from the end, as in FIG. 1A. It will appreciated that the sensors 10 may be located at different positions along the length of the tube 12. More than four sensors and wires can also be used.

In a preferred embodiment, the sensors 10 are positioned along the tube at varying lengthwise positions. In other words, when the tube 12 is inserted into an opening in the skull, the sensors 10 are positioned at varying depths within the brain tissue. For example, in a procedure where the tube 12 is inserted ten centimeters into the brain, the twelve o'clock sensor 10 extends two centimeters into the skull, the three o'clock sensor 10 extends four centimeters into the skull, the six o'clock sensor 10 extends six centimeters into the skull and the nine o'clock sensor 10 extends eight centimeters into the skull. It will be appreciated that the dimensions given herein are only exemplary, and the sensors can be positioned lengthwise at different intervals than those described herein. In another embodiment, the sensors 20 can all be positioned circumferentially around the outside of the tube at the same length along the tube 12.

In use, in order to reach the ventricle with the end of the tube 12, a doctor must traverse the normal brain. Therefore, during or after this procedure, the probes/wires can be actuated to sense the oxygen partial pressure in the brain tissue. The wires 22 are in electrical communication with a monitor 30 to which the results are output and displayed. For example, the results can be communicated to a monitor, such as an ICU monitor and displayed in wave form or other type of data. In a preferred embodiment, the catheter can also read intracranial pressure. The intracranial pressure readings can be shown on the same monitor as the tissue oxygenation data or on a separate monitor.

As shown in FIGS. 3-4, in another embodiment, the wires 22 can be configured so that the ends thereof can extend outwardly at an angle (e.g., 90 degrees) so that the sensor 10 extends further into the brain matter to get a reading. In a preferred embodiment, wires 22 each include an end portions 22a that is configured to be deployable or movable between an engaged position and a disengaged position. Therefore, when the tube 12 is inserted, the end portions 22a of the wires 22 and sensors 10 are in the disengaged position, but when a reading is to be taken with a specific sensor (e.g., the three o'clock sensor 10 in FIG. 4), the end portion 22a of the wire 22 and the sensor 10 move from the disengaged position to the engaged position. Any method of moving the end portion 22a of the wire 22 and the sensor 10 from the disengaged position to the engaged position is within the scope of the present invention. For example, a lever mechanism can be used or a switch or switches that are in electrical communication with the end portions 22a of the wires 22 can be used.

In another embodiment, there can be a sensor within the tubing, as opposed to the wires. In another embodiment, the oxygen partial pressure in the brain tissue can be sensed chemically. Any type of technology for monitoring or determining oxygen partial pressure in the brain tissue is within the scope of the present invention. In an embodiment, the device can also include the ability to measure the intracranial pressure, similar to the Hall and Millar patents listed above in the Background of the Invention section.

In another embodiment, the sensors 10 can be used for measuring things other than oxygenation content. For example, the sensors can be used to measure intracranial pressure, ph, acidity, drug levels and other desirable data. In a preferred embodiment, at least a portion of the catheter, e.g., the end of the tube and the sensors can be covered with an antibiotic coating. Furthermore, in a preferred embodiment, the catheter is disposable.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed, at different times. Further any specific numbers noted herein are only examples: alternative implementations may employ differing values or ranges.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A catheter for draining body fluid, the catheter comprising:
a tube for draining fluid, wherein the tube has an outside surface, an inside surface, a tip and defines a lumen therein, wherein the tip includes at least one opening therein that is in fluid communication with the lumen, at least a first sensor positioned on the outside surface of the tube at a first location, wherein the first sensor is configured to measure the oxygen partial pressure in brain tissue, at least a first wire having a proximal end and a distal end, wherein the distal end is in electrical communication with the first sensor and the proximal is configured to be in electrical communication with a monitor, wherein the tube and the first sensor can be inserted into a single hole in a patient's cranium.

2. The catheter of claim 1, further comprising a second sensor positioned on the outside surface of the tube at a second location, wherein the second sensor is configured to measure the oxygen partial pressure in brain tissue, a second wire having a proximal end and a distal end, wherein the distal end is in electrical communication with the second sensor and the proximal end is configured to be in electrical communication with a monitor.

3. The catheter of claim 2 wherein the outer surface of the tube has a circumference, and wherein the second location is between approximately 10° and approximately 180° from the first location about the circumference of the outer surface of the tube.

4. The catheter of claim 3 further comprising a third sensor positioned on the outside surface of the tube at a third location, wherein the third sensor is configured to measure the oxygen partial pressure in brain tissue, a third wire having a proximal end and a distal end, wherein the distal end is in electrical communication with the third sensor and the proximal end is configured to be in electrical communication with a monitor.

5. The catheter of claim 4 wherein the third location is between approximately 10° and approximately 180° from the first location about the circumference of the outer surface of the tube.

6. The catheter of claim 5 further comprising a fourth sensor positioned on the outside surface of the tube at a fourth location, wherein the fourth sensor is configured to measure the oxygen partial pressure in brain tissue, a fourth wire having a proximal end and a distal end, wherein the distal end is in electrical communication with the fourth sensor and the proximal end is configured to be in electrical communication with a monitor.

7. The catheter of claim 6 wherein the second location is approximately 90° from the first location about the circumference of the outer surface of the tube, wherein the third location is approximately 90° from the second location about the circumference of the outer surface of the tube, and wherein the fourth location is approximately 90° from the third location about the circumference of the outer surface of the tube.

8. The catheter of claim 3 wherein the first location is a first distance from the tip, and the second location is a second distance from the tip, and wherein the first distance is different than the second distance.

9. The catheter of claim 5 wherein the first location is a first distance from the tip, the second location is a second distance from the tip, and the third location is a third distance from the tip, wherein the first, second and third distances are all different.

10. The catheter of claim 7 wherein the first location is a first distance from the tip, the second location is a second distance from the tip, the third location is a third distance from the tip, and the fourth location is a fourth distance from the tip, wherein the first, second, third and fourth distances are all different.

11. The catheter of claim 1 wherein the first wire is secured to the outside surface of the tube.

12. The catheter of claim 1 wherein the first wire is embedded in the tube, and wherein the first sensor extends outside of the tube.

13. The catheter of claim 1 wherein the first wire includes an end portion that is movable between a disengaged position where it is positioned adjacent the outer surface of the tube and an engaged position wherein it extends outwardly away from the outer surface of the tube.

14. The catheter of claim 7 wherein each of the first, second, third and fourth wires include an end portion that is movable between a disengaged position where it is positioned adjacent the outer surface of the tube and an engaged position wherein it extends outwardly away from the outer surface of the tube.

15. A method of draining fluid through a single opening in a patient's cranium, the method comprising the steps of:
providing a catheter that includes a tube and at least a first sensor for measuring oxygen partial pressure in brain tissue,
inserting the tube into the opening in the patient's cranium such that the first sensor is surrounded by the patient's brain tissue,
draining fluid from the opening, and
measuring the oxygen partial pressure of surrounding brain tissue.

16. The method of claim 15 wherein the first sensor includes a first wire in electrical communication therewith, and wherein the first wire extends through the opening in the patient's cranium.

17. The method of claim 15 wherein the catheter includes a second sensor and a second wire in electrical communication therewith, and wherein the second wire extends through the opening in the patient's cranium.
